(19) United States
(12) Patent Application Publication
(10) Pub. No.: US 2012/0116329 A1
(43) Pub. Date: May 10, 2012

(54) Sealing Device For Vacuum Pressure Therapy

(76) Inventor: Juan Marquez Cañada, Valencia (ES)

(21) Appl. No.: 13/140,784

(22) PCT Filed: Sep. 30, 2010
(86) PCT No.: PCT/ES2010/000337

§ 371 Date: Jun. 17, 2011

(30) Foreign Application Priority Data
Jul. 30, 2009 (ES) P200901747

(51) Int. Cl. A61M 1/00

(52) U.S. Cl. 604/319

(57) ABSTRACT

A sealing device for vacuum pressure therapy, including a film that includes a section (5) of a tube fixed or prefixed through a sealed union (6), and able to connect to the suction tube (4), having at its periphery an adhesive area (7) covered by protective flaps (8). In an alternative variant, the device has a piece of foam (9) built inside.

### OBJECT OF THE INVENTION

The invention, as expressed in the title of this specification, refers to a sealing device for vacuum pressure therapy, contributing to the function for which is intended various advantages and characteristics of novelty, which are an improved alternative to currently known systems for the same purpose.

More particularly, the object of the invention focuses on a sealing device intended to allow the application of vacuum pressure therapy in bodily injury, especially surgical wounds, which presents the particularity of having an innovative structural design that substantially improves its effectiveness and performance.

### FIELD OF APPLICATION OF THE INVENTION

The field of application of the present invention is part of the technical sector of the industry involved in the manufacture of dresings and sealing devices applied to wounds.

### BACKGROUND OF THE INVENTION

The application of vacuum pressure on a wound bed has been demonstrated both experimentally and clinically, which accelerates tissue repair.

In general, the system for applying vacuum therapy involves placing a gauze or reticulated polyurethane foam with large open pores covered with an adhesive film and covering the entire surface of the wound. On this dressing (understood as the set of foam or gauze plus the adhesive film that covers and attaches to the surrounding skin) a suction tube that connects to a programmable vacuum pump is attached. The vacuum pump is equipped with a reservoir for storing fluid that is extracted from the wound. The dressing and the exit of the tube are sealed and isolated from the outside, creating a closed system. The high porosity of the dressing used by this system means that the pressure that is distributed across the wound surface is uniform, for this, it is imperative to ensure that the system is airtight and there are no leaks through the insulating plastic dressing.

In fact, at the very beginning, the system connecting the tube to the dressing was done in a traditional way by inserting the end of the tube into the dressing and securing it at least by two adhesive strips that overlapped each other (called “ties”) and which constituted the main source of failure of vacuum systems due to leakage of air between the tube and these “ties.”

At present, the sealing connector device more effective in preventing these leaks is manufactured by KCI (San Antonio, Tex.) And comprises an integral connector tube leading to a flexible plate which is secured to the dressing over the wound to be treated by means of an adhesive film incorporated in this plate. That is, it would a device “tube-plate-adhesive film.”

However, if it is true that the application of vacuum therapy on the body surface generates negative pressure by the pores of the dressing, the fact remains that in terms of the solid components of the device, the system generates positive pressure.

In fact, there is the potential risk to decrease the vascularity in the area under the plate, so that, when applied to ulcers or wounds of a diameter less than the one of the plate, the system has caused sometimes decubitus ulcer (i.e. ischemia by positive overpressure) on perilesional soft tissues.

Therefore, the sealing system has been modified recently to reduce its adverse effects, so that, currently, the plate has been reduced in diameter and is a little more flexible. However, “the problem”, i.e. the plate persists and, moreover, is still not applicable because of its excessive size (4 cm in diameter) in many areas of the body.

It is therefore the object of this invention to provide to the state of the art a new sealing device for vacuum pressure therapy that can effectively remedy the disadvantages described above, should be noted that, the applicant is not aware of any other invention presenting technical, structural and constitutive characteristics similar to those presented by the one here recommended.

### EXPLANATION OF THE INVENTION

Thus, the sealing device for vacuum pressure therapy that the present invention proposes is configured as a remarkable novelty in its field of application, being the characterizing details that distinguish it from what is already known in the market, adequately reflected in the concluding claims that accompany this specification.

Concretely, according to a first embodiment of the device of the invention, this, instead of being flat and needing to be secured by an adhesive film, is a piece of concave configuration and elastic material so that, when connecting a suction tube to produce a vacuum in its central area, it remains attached to the surface to be treated by a suction effect. That is, it is a “tube-cupping” device that has no additional complementary adhesive films, with the inherent advantages that entails: faster implementation, ability to withdraw or modify the location of the device after placement.

Furthermore, in a second embodiment of the invention, said concave part is replaced by a film, which is fixed to the drainage tube without intermediate plates. This film (which may be of the same or similar material to the adhesive films that are part of the conventional vacuum dressings) is fixed or prefixed from manufacture to the drainage tube (unlike the traditional fixation with “ties” of the primitive devices) to prevent leakage and its periphery being adhesive, having protective flaps that are removed at the time of use to fix the device to the dressing placed on the wound.

That is, it is a “tube-film” adhesive device in which the drain tube would be a section of suction tube fixed or prefixed to it by a sealed union, said segment being able to connect for example by means of snap on pressure to the suction tube that connects to the pump to produce vacuum, being said section of a material similar to that of redon tubes or tubes of conventional vacuum systems (latex, silicone, . . . ).

In addition, optionally, factory made or prefabricated foam may be incorporated, resulting advantageously interesting in the case of loss of substance of reduced diameter which facilitates faster placement.

On the other hand, the recommended configuration of the device described, in the case it consists of a “tube-film”, advantageously allows embodiment in very small sizes, allowing its application and adaptation to any body area, as does not include sealing plates that limit its application.

The described sealing device for vacuum pressure therapy, therefore, represents an innovative structure of constituent and structural characteristics previously unknown to date for such purpose, reasons that linked to its practical usefulness, endow it with sufficient grounds to obtain the privilege of exclusivity requested.

### DESCRIPTION OF THE DRAWINGS

To complement the description being made and in order to help better understand the features of the invention, a set of drawings is attached to the present specification as an integral part of itself, wherein the following is shown as way of illustration but not limited to:

FIG. 1. —Shows an elevation view of an embodiment of the sealing device for vacuum pressure therapy object of the invention, showing its conical body which allows its fixing by vacuum effect.

FIGS. 2 and 3.—Show both views in elevation and bottom perspective view respectively of the device in its adhesive variant.

FIG. 4.—Shows an elevation view of another example of the device, according to the invention, with the foam incorporated.

### PREFERRED EMBODIMENT OF THE INVENTION

In view of the aforementioned figures, and according to the numbering adopted, it can be seen in them a preferred embodiment of the invention, which comprises the parts and elements listed and described in detail below.

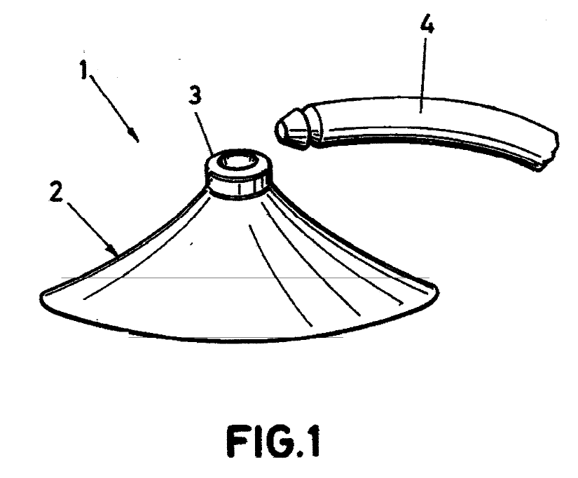

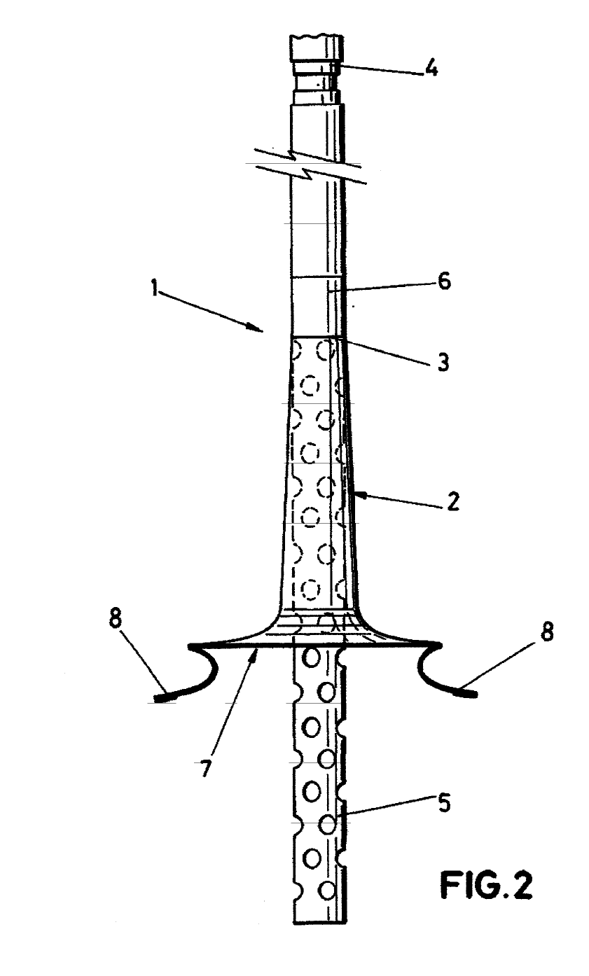

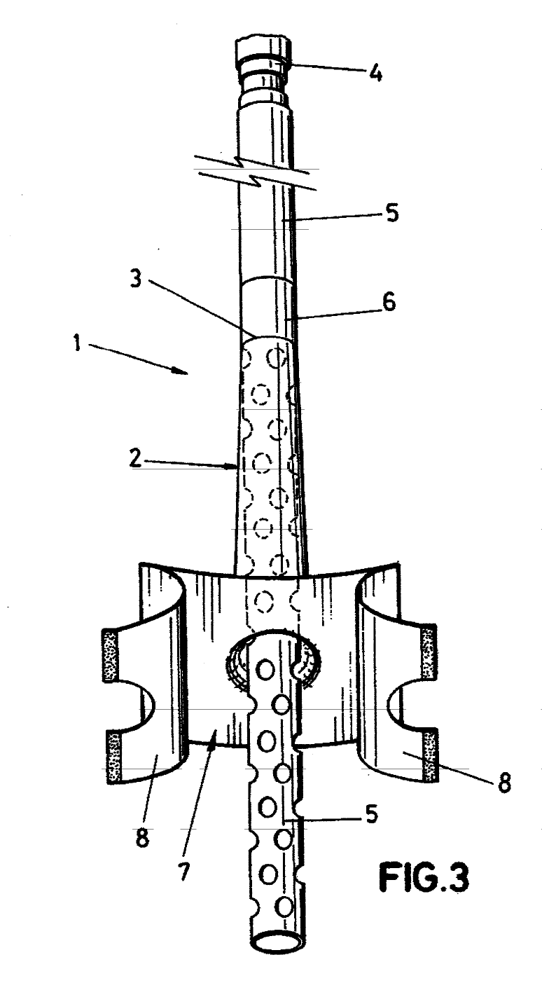

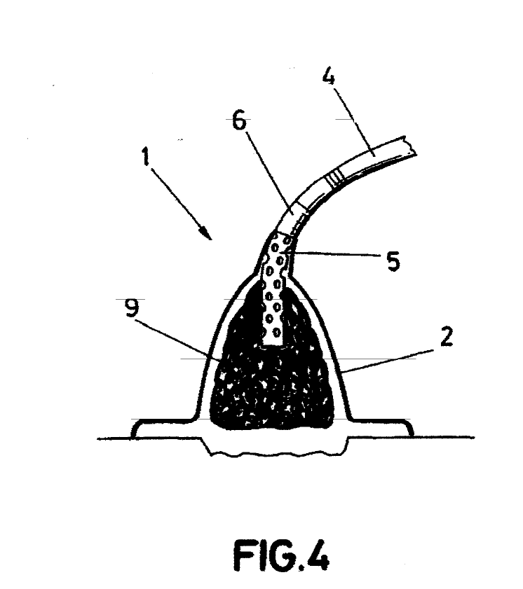

Thus, as shown in FIG. 1, the device (1) in question, which as mentioned is a sealing device for vacuum pressure therapy of the type comprising the application, on a dressing covering the wound to be treated, of a suction tube that connects to a programmable vacuum pump, comprises a part (2) of concave configuration and elastic material suitable to be connected, in its central area (3), for example through snap on pressure, to a suction tube (4) to produce the vacuum, said part (2), thanks to said concave configuration, is fixed to the surface to be treated by a suction effect, without the need to incorporate adhesive film.

In a variant of an alternative embodiment of the invention, said concave part (2) is replaced by a film, which incorporates a section (5) of suction tube fixed or prefixed to it through a sealed union (6), being said section (5) able to connect, for example by snap on pressure to the suction tube (4) that connects to the pump to produce vacuum.

In this variant of the embodiment, the film (2) has at its periphery, an adhesive area (7) covered by protective flaps (8) that are removed at the time of its use to be able to fix the device to the dressing placed on the wound.

Optionally, there is a possibility that the device (1), either factory made or prefabricated, has a portion of foam (9) built inside.

Sufficiently described the nature of the present invention, as well as how to implement it, it is not considered necessary to extend its explanation for any person skilled in the art to understand its scope and the benefits that derive from it, stating that, within its essentiality, it can be put into practice in other embodiments that differ in detail from that indicated by way of example, and to which the protection claimed will also reach them provided that its fundamental principle is not altered or modified.

1-3. (canceled)

4. A sealing device for vacuum pressure therapy, the kind that comprises the application, over a dressing covering the wound to be treated, of a suction tube that is connected to a programmable vacuum pump, characterized in that it comprises a film that incorporates a section (5) of a suction tube fixed or prefixed to it through a sealed union (6),being said section (5) able to connect, for example by snap on pressure to the suction tube (4) that is connected to the pump to produce vacuum, having the film (2) at its periphery an adhesive area (7) covered by protective flaps (8).

5. The sealing device for vacuum pressure therapy according to claim 1, characterized in that the device has a piece of foam (9) built inside.
